(19) United States
(12) Patent Application Publication
(10) Pub. No.: US 2010/0075416 A1
(43) Pub. Date: Mar. 25, 2010

(54) Growth Factor Supplement for Stem Cell Cultures

(75) Inventor: Thomas Primiano, Monona, WI (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: SHILOH LABORATORIES, INC., Monona, WI (US)

(21) Appl. No.: 12/343,346

(22) Filed: Dec. 23, 2008

Related U.S. Application Data
(60) Provisional application No. 61/099,195, filed on Sep. 22, 2008.

(51) Int. Cl. C12N 5/08; C12N 5/00

(52) U.S. Cl. 435/366; 435/405

(57) ABSTRACT

Disclosed is a growth factor supplement for stem cell culture media, stem cell culture media supplemented with the growth factor supplement, and methods for growing and maintaining stem cells in culture. The invention particularly relates to human stem cells, more particularly human embryonic stem cells, neonatal stem cells, adult stem cells, and IPS cells.

This application claims priority to U.S. provisional application Ser. No. 61/099,195, filed Sep. 22, 2008, the entirety of which is incorporated by reference herein.

### TECHNICAL FIELD

The invention relates to stem cell lines useful in the therapeutic relief of disease by replacing the function of diseased native cells. This invention pertains to stem cells of any origin, embryonic, fetal or adult. For this invention, compositions of combinations of growth factors that aid stem cell growth are provided. Shiloh Laboratories has discovered, six protein factors, when combined at a concentrations of 1 to 10 ng/ml, an extremely low concentration, stimulate the growth of human embryonic stem cells (hESCs) by better than 2-fold. Plating efficiencies are greatly enhanced using ProSen™. Preliminary results indicate that cells can be maintained without noticeable differentiation for three days without changing the media.

### BACKGROUND OF THE RELATED ART

Human embryonic stem cells (hESCs), because of their unique combination of long-term self-renewal properties and pluripotency, are providing new avenues of investigation of stem cell biology and human development and show promise in providing a new source of human cells for transplantation therapies and pharmaceutical testing. Current methods of propagating these cells using combinations of mouse fibroblast feeder cultures and bovine serum components are inexpensive and, in general, useful. However, the systematic investigation of the regulation of self-renewal and the production of safer sources of cells for transplantation depends on the elimination of animal products and the use of defined culture conditions. Both goals are served by the development of serum-free culture methods for hESCs.

Utilizing traditional culture conditions established for the derivation of mouse embryonic stem cells (mESCs) [1, 2], derivation of hESCs from cultured human blastocycts was achieved in the presence of both mouse embryo fibroblasts (MEF) and bovine serum [3, 4]. These pluripotent cells were shown to be capable of both unlimited self-renewal and differentiation into all somatic cell types. This discovery not only provided the technical framework from which other groups performed similar derivations (reviewed in [5]), but also set in motion numerous studies exploring various aspects of stem cell biology including self-renewal, human development, and in vitro directed differentiation. To effectively explore these avenues, it has become apparent that better-defined, serum-free growth conditions are essential. These conditions can provide a defined system by which reproducible and methodical analyses can be performed. In addition, this approach provides the potential to generate clinically relevant hESC-derived cells for therapeutic applications.

Serum-free culture systems. The culture conditions used for the initial derivation of hESCs included culture medium containing high levels (20%) of fetal bovine serum (FBS) on top of a layer of mitotically inactivated MEF [3]. As FBS is the serum of a developing calf collected from animals produced for agricultural purposes, this reagent is by nature a variable in its components, often resulting in significant lot-to-lot variability. In addition, animal-derived components can introduce known or unidentified animal pathogens into hESC cultures, an undesirable factor for downstream therapeutic applications [6]. To avoid this problem, efforts have been made to test the ability of adult human serum (HS) to support the hESC culture. One study reported the ability to extensively (more than 40 passages) propagate a hESC line in HS-containing medium with continual self-renewal, maintained pluripotency and a stable diploid karyotype, with continual propagation of undifferentiated cells for more than 40 passages [7]. Although HS provides an animal-free serum alternative, this source, much like FBS, is plagued by batch variability, poorly defined compositions, and variable efficacies in hESC cultures [8-10]. Studies have been done in other stem cell systems to identify some of the absolutely essential serum-provided components including albumin, transferrin, and insulin [11, 12]. A major advancement in the establishment of a serum alternative was the development of a commercially available serum replacement referred to as Knockout Serum Replacement (KSR, Invitrogen) [13]. Human ESCs derived under serum-containing conditions can be transitioned to KSR-containing media without an observed loss of long-term self-renewal or pluripotency [14], and with an enhanced ability to clonally passage hESCs (albeit low, <1%). Further, hESCs cultured in KSR-containing medium have an increased growth rate compared with those grown in FBS-containing medium [8]. Other groups demonstrated the ability of this supplement to support derivation of novel hESC lines [15-17]. To further understand the mechanisms supporting the enhanced growth of hESCs in KSR-containing medium, Skottman et al. [18] examined the gene expression profiles of hESCs cultured under FBS-containing and KSR-containing media formulations. This study demonstrated that both conditions resulted in morphologically undifferentiated cells with similar expression profiles of pluripotency associated genes (i.e. Oct4, Nanog, Cripto and DNMT3B) and their downstream signaling partners, suggesting a nonpluripotency related cause for enhanced growth in serum replacement. However, many differentially expressed genes in medium containing serum included those expressed in differentiated cells, with cluster analysis revealing a greater similarity between fibroblasts and hESCs cultured in serum than with hESCs cultured in KSR. As 40% of the shared but differentially expressed genes in this study have no known biological function, further studies are required to clarify the basis for these observations. Although KSR was designed to provide more standardized and better defined supplements to ESC media, it contains Albu-MAX, a lipid-rich albumin fraction of bovine serum, and is therefore not free of animal-derived components [13, 19]. Recently, commercially available animal-free serum replacement products have been compared for their utility in hESC culture, including Lipumin, SerEx (PAA Laboratories), Serum Replacement 3 (Sigma), Serum Substitute Supplement (Irvine Scientific), and Plasmanate (Bayer Healthcare). Although these products have theoretical promise as medium supplements, suboptimal results were obtained compared with medium containing KSR [10]. A refinement of medium supplements based on known factors influencing hESC self-renewal and differentiation may enhance these systems.

Supplementation of hESC culture systems. Numerous studies have been performed to test various cytokine supplements in support of ESC maintenance. Early studies in the mouse system found that an interleukin-6 class cytokine, leukemia inhibitory factor (LIF), was able to maintain undifferentiated ESC growth, in serum-containing media in the absence of MEFs [20]. This molecule, upon binding to a heterodimeric receptor composed of the LIF receptor and gp130, activates Janus Kinase (JAK)/Signal Transducers and Activators of Transcription 3 (STAT3) signaling. In addition, it was discovered that the addition of BMP4 to LIF-containing serum-free medium has a synergistic effect on mESC self-renewal by inducing expression of Inhibitor of Differentiation (Id) genes [21]. While these studies provided the basis for understanding the regulation of mESC self-renewal through the inhibition of differentiation of specific cell lineages, these cytokines do not have the same effect on hESC self-renewal. In contrast to mouse ESCs, the maintenance of the undifferentiated growth of hESCs occurs independent of STAT3 activation [3, 22-26]. As hESCs were shown to be able to be grown feeder-free through the use of MEF-conditioned medium and an appropriate attachment substrate (i.e. Matrigel-coated or laminin-coated culture dishes) [27, 28], it became possible to systematically define and characterize both the required exogenous factors and the factors produced by supportive feeder-layer cells. Of the factors tested, basic FGF (bFGF) is the most commonly used medium component required for hESC maintenance, both in the presence and the absence of fibroblast feeders [27]. High concentrations of bFGF can support long-term, feeder-independent self-renewal of hESCs when combined with noggin, an inhibitor of BMP signaling activity [24]. To clarify the role bFGF plays in hESC cultures, Greber et al. performed stimulation experiments followed by microarray gene expression profiling on both MEFs and undifferentiated hESCs [29]. A subtractive analysis compared the expression profile of secreted proteins with both supportive and non-supportive feeder-layer cells. Stimulation of MEFs with bFGF ultimately resulted in the regulation of key members of the transforming growth factor beta (TGFb) pathway, including upregulation of Inhba (activin A), Tgfb1 and Grem1 as well as down-regulation of Bmp4. Similarly, bFGF restimulation of feeder-free hESCs revealed regulation of a strikingly similar group of genes, suggesting that regulation of TGFb signaling is one of the important roles bFGF plays in hESC self-renewal. In hESCs the response to bFGF revealed an overrepresentation of genes involved in cell cycle regulation (i.e. CDKN2B and GADD45A) and transcription factors (i.e. Nanog and FoxD1). Recent evidence suggests that other supplemental factors to hESC media can enhance hESC cultures, including TGFb and LIF [19] activin A, keratinocyte growth factor (KGF), nicotinamide [30] and sphingosine-1-phosphate (S1P)/platelet-derived growth factor (PDGF) [31]. In support of these observations, the roles of TGFb/activin/nodal and FGF signaling pathways have been further characterized in hESCs [32, 33]. It is noteworthy that while some supplemental factors (i.e. Activin) appear to affect hESC culture by modulating self-renewal and maintaining the undifferentiated state of hESCs, others appear to serve as mitogens (i.e. KGF), inducing proliferation [30]. Three Toll-like receptor kinase ligands, brain-derived neurotrophic factor, neurotrophin 3 and neurotrophin 4, are survival factors for hESCs. Addition of neurotrophins to hES cell cultures effects a 36-fold improvement in their clonal survival [34]. The survival activity of neurotrophins in hESCs is mediated by the phosphatidylinositol-3-kinase pathway but not the mitogen-activated protein kinase pathway. Promising advancements have also been made in the field of chemical biology, whereby cell-based phenotypic and pathway-specific screens have been done to identify synthetic small molecules and natural products capable of controlling cellular processes (for reviews see [35-37]). Enhancers of ESC self-renewal, including PD98059, a MEK inhibitor [38]; 6-Bromoindirubin-30-oxime (BIO), a Wnt signaling activator [39]; SC1, an inhibitor of ERK1-dependent and RasGAP-dependent signaling pathways [40]; and more recently IQ-1, a modulator of Wnt/b-catenin/CBP signaling [41] have been found. While some data have been presented on short-term hESC maintenance [39], future efforts are necessary to validate the roles these molecules can play in long-term hESC cultures.

Feeder-free and human-feeder layer hESC cultures. Much like serum, the use of supportive feeder cultures has been fraught with concerns. As MEFs are a primary culture population derived from fetal mouse tissue, batch variability is a problem encountered by many labs. In addition, the use of MEFs incorporates an animal-derived component to hESC cultures, a concern for potential downstream hESC therapies. This concern was validated when it was observed by Martin et al. [42] that hESCs cultured in KSR-containing media and/or with MEFs incorporate substantial amounts of N-glycolylneuraminic acid (Neu5Gc), a mammalian sialic acid displayed on the surface of non-human mammalian cells. As humans are genetically unable to produce Neu5Gc, transplanted cells displaying this antigen would be recognized as foreign and destroyed by an immune response, although culture modification or differentiation greatly reduced or eliminated the presence of Neu5Gc on hESCs [42, 43]. As it was shown that hESCs could be continually propagated on animal-free sources including human feeder-layer cells [9, 17, 44-49], autogeneic hESC-derived feeder-layer cells [49], and various feeder-free matrices [19, 27, 30, 50, 51] (for a review see [52]) completely defined animal-free culture systems are actively being pursued.

Animal-free defined culture systems. Early defined, feeder-free hESC cultures utilized Matrigel, a solubilized basement membrane mixture secreted by mouse sarcoma cells. While this matrix sustained undifferentiated growth of hESCs in conditioned or growth factor supplemented media [24, 53], efforts were made to utilize better defined matrices including laminin [30, 53] and fibronectin [19, 30]. Since these culture systems utilized bovine albumin-containing KSR in their medium, additional refinements were necessary to generate a truly humanized defined medium. As proof of principle, hESCs were derived and maintained in a feeder-free, defined medium (TeSR1) that included protein components derived from only recombinant or purified human sources [43], including human serum albumin, vitamins, antioxidants, trace mineral, lipids and a specific list of cloned growth factors (bFGF, LiCl, g-aminobutyric acid (GABA), pipecolic acid, and TGFb). The medium components were selected from a relevant list screened with hESCs on Matrigel-coated dishes. Further analysis demonstrated that this medium could support long-term culture of hESCs when grown on a matrix containing recombinant human collagen IV, fibronectin, laminin, and vitronectin, and that these conditions allowed the derivation of two novel hESC lines. This work not only brought a great deal of theoretical promise to therapeutic applications of hESCs and is the medium used for derivation of induced pluripotent stem (iPS) cells [54].

The use of defined conditions using recombinant human reagents is the ultimate goal for studies aimed at understanding the regulation of hESC self-renewal and the generation of therapeutically useful hESC-derived cell products. Promisingly, much progress has been reported in the several years since hESCs were first described. Further progress in identifying defined culture conditions that maintain undifferentiated hESC, maximize proliferation rates, maintain normal karyotypes, and improve the efficiency of subcloning these cells must be made. Finally, the cost of this sequentially more defined approach becomes prohibitive for routine laboratory use. As a result, the Thomson lab has reported a modified version of their defined medium (mTeSR1) that includes the use of animal source proteins (bovine serum albumin and Matrigel) and cloned zebrafish bFGF [55], a more practical formulation for routine research-based hESC culture. To make the use of defined, humanized systems practical for routine research or clinical applications, lower cost alternatives are necessary. This may be achieved as simpler media [56-58], defined supplements, and synthetic substrates [57, 58] are tested for hESC cultures.

### Usefulness of Stem Cells.

Results obtained from studies with mouse ESCs (mESCs) raise the possibility that clinical trials with hESCs are not far off. mESCs have been manipulated to become spinal cord motor neurons [59], dopaminergic neurons [60, 61], and many other types of cells. One example must suffice here to emphasize their therapeutic promise. In one of the most thorough and elegant studies published to date, mESCs were induced to differentiate into spinal cord motor neurons by successive exposure to retinoic acid and sonic hedgehog, a protein known to trigger the differentiation of motor neurons in developing embryos [59]. When treated cells were injected into the spinal cord of a chick embryo, they migrated to their proper location in the ventral horn. Some cells sent axons out of the spinal cord to invade the developing limb and form synapses on target muscle fibers. This type of research lends hope to individuals suffering from amyotrophic lateral sclerosis, spinal muscular atrophy, spinal cord injury, and related disorders.

Pancreatic islets have been implanted into patients with type 1 diabetes to restore them to insulin independence (Ricordi, C. and T. B. Strom, CLINICAL ISLET TRANSPLANTATION: ADVANCES AND IMMUNOLOGICAL CHALLENGES, Nat. Rev. Immunol. 2004, 4(4): 259-268). Islet transplantation is successful, when performed according to the Edmonton protocol [61, 62]. Likewise, implantation of fetal mesencephalic brain tissue into the brains of patients with Parkinson disease has resulted in measurable improvement in some indices of motor performance [63]. Both implantation studies, however, were limited by tissue availability and, in the Parkinson disease study, there were serious side effects (e.g., dyskinesias). Both studies call for further work with hESCs, with the hope of moving to Phase 1 clinical trials.

There is much to learn regarding the use of stem cells for the treatment of disease. One present limitation on using hESCs for treating disease is that they must be grown on feeder layers, heterologous non-stem cells that produce factor(s) necessary for normal hESC growth. These cells are conventionally irradiated mouse embryonic fibroblasts, which function quite reliably in vitro. However, xenobiotic concerns such as mouse retrovirus contamination are a serious drawback to using hESC grown on such murine feeder layers. There is thus a need in the art to identify human cells that can be used as feeder layers in the same way, but without the xenotoxicity concerns. No such human cell has been identified to date.

Also needed in the art is additional information about how to keep ESCs dividing until they are called on to differentiate. Such needed knowledge includes information on growth factors that influence hESC differentiation into diverse cell types. Most importantly, stem cell therapy protocols are needed that are safe as well as effective. This will be greatly facilitated by understanding how to inhibit proliferation and/or metabolic activity of these cells off in vivo in the event that toxicity develops. In addition, the risk of immune rejection remains a problem. Given the limited genetic diversity of available cell lines, transplantation of stem cell products can be expected to be subject to the same immune barriers as organ transplantation. At the present time, the only defense against rejection is the administration of long-term immunosuppression therapy, which increases the patients' risk of infection and is associated with nephrotoxicity.

### SUMMARY OF THE INVENTION

This invention provides a combination of growth factors that supports growth of stem cells, particularly human stem cells and specifically human embryonic stem cells, under conditions that avoid the need for a cellular feeder layer. The invention thus provides reagents and methods for growing stem cells, particularly human stem cells and specifically human embryonic stem cells, under feeder cell-free conditions that thus avoid xenobiotic contamination concerns. The invention also provides a chemically-defined media for growth on stem cells, particularly human stem cells and specifically human embryonic stem cells.

As provided herein, stem cell growth media comprises conventional cell culture media, preferably mTeSR1™ media (BD Biosciences, Bedford, Mass.), supplemented with a plurality of specific growth factors. In one embodiment, said combination of growth factors comprises human interleukin 5, human interleukin 6 and human interleukin 7. In another embodiment, said combination of growth factors comprises human interleukin 5, human interleukin 6, human interleukin 7 and human granulocyte macrophage colony stimulating factor (GM-CSF). In another embodiment, said combination of growth factors comprises human interleukin 5, human interleukin 6, human interleukin 7, human GM-CSF and human GRO gamma/CXCL3 [64]. In another embodiment, said combination of growth factors comprises human interleukin 5, human interleukin 6, human interleukin 7, human GM-CSF, human GRO gamma/CXCL3 and CCL2/MCP-1 [65]. As provided herein, in each of said embodiments each of said growth factors are supplied at a concentration of at least about 1 ng/mL. Each of said growth factors is commercially available.

### BRIEF DESCRIPTION OF THE DRAWINGS

FIG. 1. Identification of growth factors secreted from FibroSen™ cells. This Figure shows the results of secreted cytokine immunoassay (left panel) and the results displayed graphically (right panel) produced as follows. A confluent cell layer was prepared by adding 50,000 FibroSen™ cells to each well, allowing the cells to reach confluence after 4 days, and then adding 10 μM IPTG to trigger differentiation in triplicate test wells. Control wells received no IPTG. IPTG was incubated on the cells for 48 hours. The media was washed from the cells with PBS. Media lacking serum was added onto the cells at day 0. Conditioned media from FibroSen™ feeder cells were collected after 3 and 5 days. Media was filtered through a 0.2 um PES filter. Supernatant proteins from either non-differentiated (lacking IPTG) or differentiated (with IPTG) cells were then incubated on triplicate cytokine antibody array membranes (Ray Biotech) overnight at 37° C. The next day, biotinylated antibodies to all of the spotted cytokines on the array were incubated with the membrane, washed then incubated with ³⁵S methionine labeled streptavidin. The membranes were washed and exposed to a phosphorimaging plate for 2 hr. Left Panel: The spots were visualized with a Fuji phosphorimager and six growth factors were identified. Right Panel: The growth factors were selected based on ratio of the intensity of the spots detected from the antibody array membrane for those samples from IPTG treated FibroSen™ cells against control. Factors with more than 2-fold increase in spot intensity after 3 days were chosen. Means were tested by t-test following ANOVA with confidence interval of p<0.05. Results from Day 3 are plotted to the left and results from Day 5 are plotted to the right in each pair of results for each growth factor.

FIG. 2. ProSen™ supplement on the growth of H9 cells in feeder free culture. The capacity of H9 human embryonic stem cells to grow in media supplemented with combinations of growth factors according to the invention is shown. H9 cells were seeded at a density of 50,000 cells in 1 ml mTeSR medium in nine separate wells in a 12 well plate coated with Matrigel. IL5, IL6, IL7, GM-CSF, GROγ, and MCP1 at 1 ng/ml each was added to three wells. At 1 ng/ml, the combination of growth factors was capable of increasing the number of H9 cells by more than 2-fold after 6 days in culture. Using 0.1 ng/ml of each growth factor had no different effect on H9 cell growth as mTeSR1 media alone. Top, left panel: H9 after 6 days in mTeSR only. Top, right panel: H9 after 6 days with 1 ng/ml ProSen™. Bottom panel: Graph showing viable cell density of H9 cells after 6 days in culture. Differences between 1 ng/ml ProSen™ against mTeSR alone and 0.1 ng/ml ProSen™ were determined significant using Scheffe's test following ANOVA, p<0.05.

FIG. 3. ProSen supplement prolongs the usable lifetime of mTeSR1 media. Cell culture plating experiments were performed to demonstrate increase in useable lifetime of cells culture in mTeSR1 media supplemented with ProSen. H9 hES cells were plated at 50,000 cells per well in 1 ml mTeSR1. The next day, ProSen™ supplement (1 ng/ml of each growth factor) was added to begin the experiment (Day 0). Viable cell density (the number of viable cells/ml) was determined at days 2, 3 and 4. Left panel: Viable cell density with ProSen™ doubled each of the first 3 days of culture without a change in media. H9 cells in mTeSR1 alone did not proliferate and began to die. Right panel: Photomicrographs display normal looking colonies of H9 cells in ProSen™ after 3 days without changing media. The results plotted in the graph are arrayed left to right as Day 2 results, Day 3 results and Day 4 results.

FIG. 4. Effects of ProSen supplement on Oct4 expression in H9 cells. H9 hES cells were plated in ProSen-supplemented mTeSR1 media and the effects on stem cell-related gene expression assayed by fluorescence-activated cell sorting (FACS) using Oct 4 antibodies. Left panel: Oct 4 immunostaining of H9 cells receiving fresh mTeSR1 media daily. Right panel: Oct 4 immunostaining of H9 cells receiving mTeSR1 media+ProSen™ supplement every 3^{rd} day. Stained cells form the peaks on the right in each graph.

FIG. 5. ProSen™ supplement enhances plating efficiency of H9 hESCs. H9 hES cells were plated in ProSen-supplemented mTeSR1 media and the effects on stem cell-related plating efficiency was assayed. H9 cells were treated with Accutase to prepare single cell suspensions. Digested cells were plated at 30,000 cells per well in 3 ml mTeSR1 on Matrigel. The next day, ProSen™ supplement (1 ng/ml of each growth factor) was added to begin the experiment (Day 0). The number of colonies was determined after 6 days in culture. Left panel: H9 plated in mTeSR1 alone did not form viable colonies. Right panel: H9 plated in ProSen™ form normal looking colonies. Note media was changed every day for this experiment.

### DETAILED DESCRIPTION OF THE INVENTION

Substantially similar results were obtained in the presence of media supplemented with a combination of growth factors each at a concentration of 10 ng/mL comprising human interleukin 5, human interleukin 6 and human interleukin 7; or with a combination of growth factors comprising human interleukin 5, human interleukin 6, human interleukin 7 and human granulocyte macrophage colony stimulating factor (GM-CSF), or with a combination of growth factors comprising human interleukin 5, human interleukin 6, human interleukin 7, human GM-CSF and human GRO gamma/CXCL3, or with a combination of growth factors comprising human interleukin 5, human interleukin 6, human interleukin 7, human GM-CSF, human GRO gamma/CXCL3 and CCL2 MCP1. Although the present invention has been described with reference to particular embodiments, it is to be appreciated that various adaptations and modifications may be made without departing from the spirit and scope of the invention. The invention is only to be limited by the appended claims.

### EXAMPLES

### Example 1

### Identification of Protein Factors that Enhance ES Cell Growth

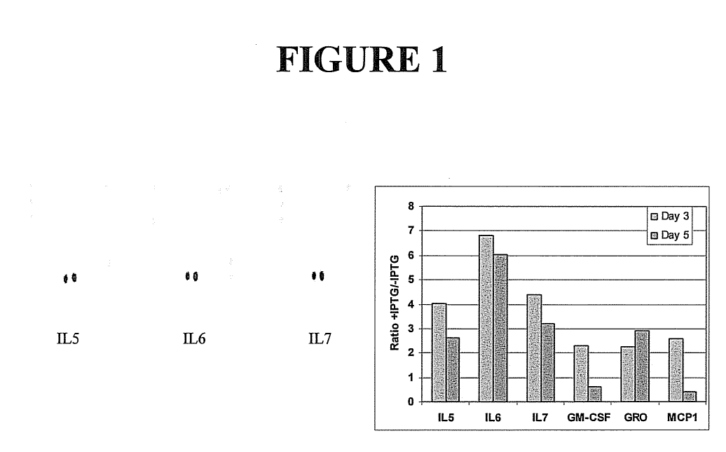

Shiloh Laboratories engineered human HT1080 fibrosarcoma cells with an inducible system that forces cell cycle arrest and terminal differentiation of the cells (called FibroSen™), as disclosed in co-owned and co-pending U.S. patent application Ser. No. 12/115,451, filed May 5, 2008 and incorporated by reference herein. Fragments of the senescence triggering protein p21 under regulation of the lactose operon promoter are repressed by constitutive expression of the lac repressor protein. Upon addition of IPTG, the repression is removed p21 factors trigger differentiation of the FibroSen™ cells. These cells provided a confluent feeder layer upon which hESCs may be plated. Not only did hESCs grow well on the FibroSen™ feeder system, but cell numbers increased dramatically, to as much as 7-fold. An intuitive hypothesis from these results is that some factors secreted from the FibroSen cells are assisting the enhanced proliferation of the hESCs. Shiloh Laboratories undertook a proteomics approach to identifying what factors are present in the medium in differentiated FibroSen™ cultures, with respect to media from non-differentiated FibroSen™ cells (FIG. 1). Media lacking serum was added to the cells and incubated for either 3 or 5 days. Supernatants from each culture were removed, centrifuged, filtered with 0.2 um PES filter. Supernatant proteins from either non-differentiated (lacking IPTG) or differentiated (with IPTG) cells were then incubated on triplicate cytokine antibody array membranes (Ray Biotech) overnight at 37° C. The next day, biotinylated antibodies to all of the spotted cytokines on the array was incubated with the membrane, washed then incubated with ³⁵S methionine labeled steptavidin. Labeled proteins were the membranes were washed and exposed to a phosphorimaging plate. Spots were visualized using a Fuji phosphorimager. The visualized spots were quantitated with the Phosphorimager software and triplicate data were averaged. Statistically significant results were used to identify 6 growth factors that had at least 2-fold higher concentrations in media from differentiated cells than control non-differentiated cells. These factors were identified as IL5, IL6, IL7, GM-CSF, GROγ, and MCP1. All of these growth factors are commercially available. The next logical experiment was to add these factors in a feeder free system to determine if they could increase the growth rates of hESCs. H9 cells were plated at a density of 50,000 cells per well in a 12 well plate coated with Matrigel.

ProSen Growth Factors. IL-5 (GF1) stimulates B cell growth and is associated with Janus kinase (JAK) 2 [66]. IL-6 (GF2) acts as a pro-inflammatory cytokine that triggers signal transduction through JAK and STAT tyrosine kinases [67]. IL7(GF3) is a hematopoietic growth factor secreted by the stromal cells capable of stimulating the proliferation of lymphoid progenitors by signal transduction through the Jak/STAT pathway [68] GM-CSF (GF4) is secreted by macrophages, T cells, mast cells, endothelial cells and fibroblasts and functions as a white blood cell growth factor by inducing protein tyrosine phosphorylation, Ras, Raf-1 and MAP kinase [69], GROγ (GF5) works through the chemokine receptor CXCR2 [70] and plays a role in inflammation. MCP1 (GF6) is a chemokine that recruits monocytes, memory T cells, and dendritic cells to sites of tissue injury and infection and is under the control of nuclear factor κB (NFκB) [71].

### Example 2

### Increased Growth Rates of hESCs with ProSen™ Supplement

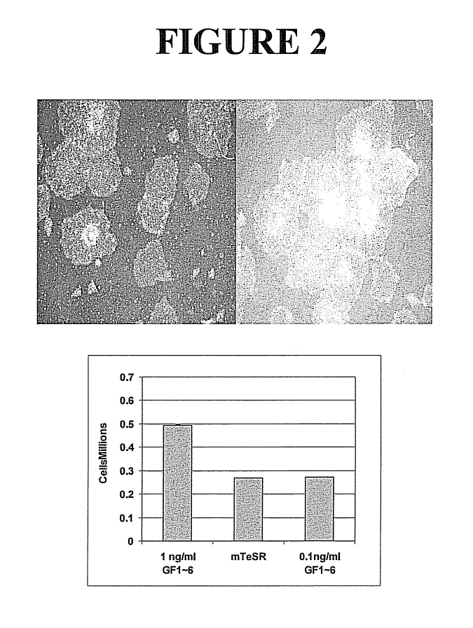

The concentration of cells was the lowest that could be used and have the H9 cells grow properly. Using a low concentration to start the experiments allows for longer experiments to be performed in the same dish. In an attempt to mimic the FibroSen™ cultures, the initial concentrations of each of the growth factors were 10 ng/ml and using all six factors at equal concentrations. For ease of discussion, the grouping of GF1-GF6 (IL5, IL6, IL7, GM-CSF, GROγ, and MCP1) was termed the ProSen™ supplement. Please note that simply adding each of the growth factors alone (i.e., individually) at 10 ng/ml concentration in mTeSR1 did not yield an increase in viable cell density of H9 cells after 6 days. These concentrations were chosen based on the effective concentration of each factor as published by the manufacturer. Using 10 ng/ml concentrations yielded more than 2-fold increase in the number of H9 cells after 6 days of culture in mTeSR1 media (Stem Cell Technologies; results not shown). The experiment was repeated with 1 and 0.1 ng/ml concentrations of the combined six growth factors. As can be seen in FIG. 2, the combination GF-1-GF6 at 1 ng/ml each was capable of increasing the number of H9 cells by more than 2-fold after 6 days in culture. Using 0.1 ng/ml of each growth factor had no different effect on H9 cell growth as mTeSR1 media alone. Media is changed every day in feeder-free culture to prevent differentiation of the cells.

### Example 3

### Extended Use of Stem Cell Media

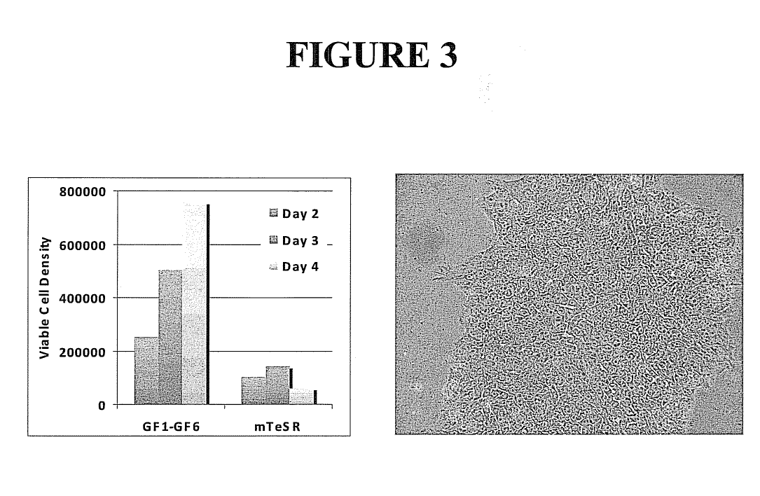

Many laboratories have unsuccessfully attempted to use growth factor supplements to extend the media life of feeder free stem cell media. To determine if ProSen™ at 1 ng/ml is capable of extending the usable lifetime of mTeSR media, cells were plated at 50,000 cells per well in 1 ml mTeSR. The next day, ProSen™ supplement was added to begin the experiment (Day 0). Viable cell density (the number of viable cells/ml) was determined at days 2, 3 and 4. Media was not changed at all during this experiment. The results of these experiments are shown in FIG. 3. Under these conditions, H9 cells in log phase were expected to double in about 30 hours. For H9 in mTeS1R, viable cell density did not double each day. In fact, the cells began to die by day 3 and the culture was filled with mostly dead cells by day 4. In contrast, using 1 ng/ml ProSen™ supplement allowed cells to continue to double for as long as 4 days without changing the media. It is suggested that using ProSen™ can lower the cost of using mTeSR1 about 6-fold by increasing the number of cells 2-fold and using 3 times less media.

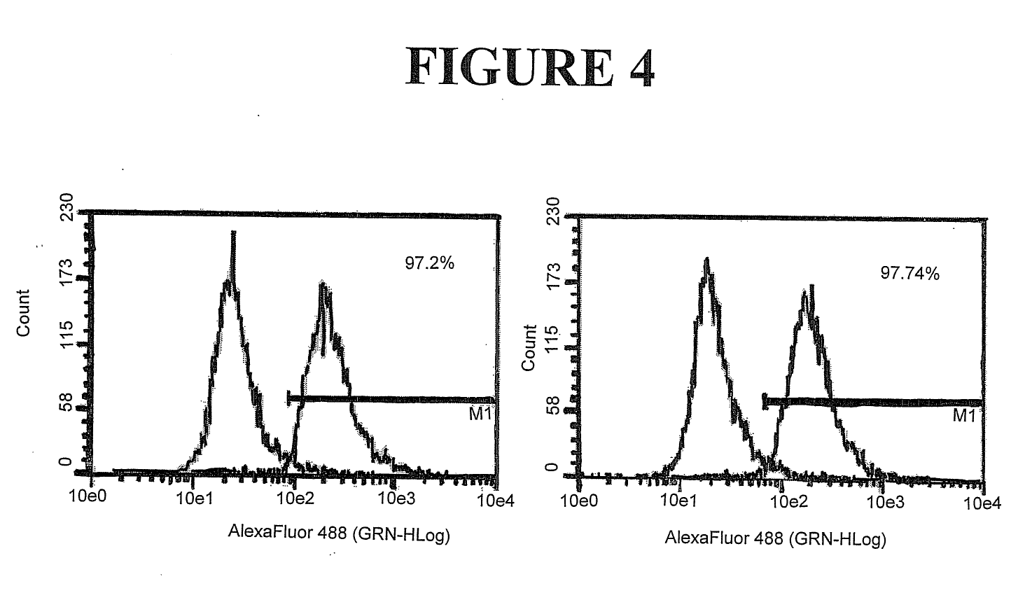

The effects of the ProSen™ supplement on pluripotency of H9 cells after 6 days in culture was determined for cells receiving mTeSR1 daily or every third day. Results of Oct 4 expression in H9 are shown in FIG. 4. More than 97% of the cells stained for Oct 4 transcription factor, a known marker of pluripotency, in both cells receiving fresh media daily or mTeSR+ProSen™ supplement every 3^{rd} day. These results indicate that under short-term exposure to ProSen™ supplement does not adversely impact pluripotency of H9 cells.

### Example 4

### ProSen™ Increases the Plating Efficiency of hESCs

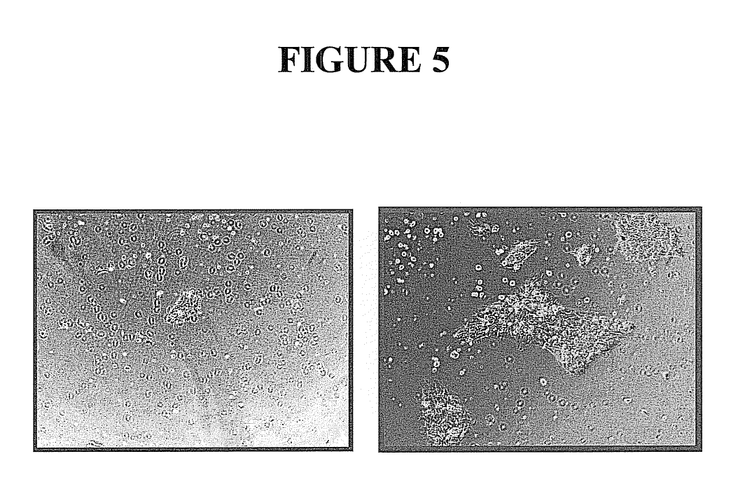

Another effort put forth by a number of laboratories using feeder-free systems for growing hESCs is to increase the plating efficiency of the cells. Plating efficiency is defined as the number of colonies that grow per cell seeded into the culture. The plating efficiency drops dramatically when lower seeding cell numbers are used. To date, it has not been possible to plate a single hESC cell in a single well of a microtiter plate and get a colony to form in a feeder-free system. Plating cells at a density lower than 3000 cells/cm² (30,000 cells/well in a 6 well plate) do not form colonies. In an attempt to determine if ProSen™ supplement can enhance the plating efficiency of H9 cells on Matrigel in mTeSR1 media, H9 cells were digested with Accutase to single cells, a process that normally does not allow for colony formation. Digested H9 cells were plated at 30,000 per well in a 6-well plate. One set of cells received the ProSen™ supplement, control cells did not. The mTeSR1 medium with or without ProSen™ was changed every day for this experiment. Colonies were counted after 6 days in culture. The results of these experiments are shown in FIG. 5. Very few and very small colonies with mostly dead cells were observed in cultures with mTeSR alone. Adding ProSen™ lead to normal colony formation with plating efficiencies of greater than 50%. Using ProSen™ and Accutase treatment may allow for true subcloning and possible automation of hESC cloning using FACS sorting, limiting dilution, or other cloning techniques that isolate single cells. Having cells of true clonal origin may aid percent conversion of hESCs to differentiation committed embryoid cells or type-specific somatic cells.

It should be understood that the foregoing disclosure emphasizes certain specific embodiments of the invention and that all modifications or alternatives equivalent thereto are within the spirit and scope of the invention as set forth in the appended claims.

### REFERENCES

1. Evans, M. J. and M. H. Kaufman, Establishment in culture of pluripotential cells from mouse embryos. Nature, 1981. 292(5819): p. 154-6.

2. Martin, G. R., Isolation of a pluripotent cell line from early mouse embryos cultured in medium conditioned by teratocarcinoma stem cells. Proc Natl Acad Sci USA, 1981. 78(12): p. 7634-8.

3. Thomson, J. A., et al., Embryonic stem cell lines derived from human blastocysts. Science, 1998. 282(5391): p. 1145-7.

4. Reubinoff, B. E., et al., Embryonic stem cell lines from human blastocysts: somatic differentiation in vitro. Nat Biotechnol, 2000. 18(4): p. 399-404.

5. Skottman, H. and O. Hovatta, Culture conditions for human embryonic stem cells. Reproduction, 2006. 132(5): p. 691-8.

6. Gruen, L. and L. Grabel, Concise review: scientific and ethical roadblocks to human embryonic stem cell therapy. Stem Cells, 2006. 24(10): p. 2162-9.

7. Ellerstrom, C., et al., Derivation of a xeno-free human embryonic stem cell line. Stem Cells, 2006. 24(10): p. 2170-6.

8. Koivisto, H., et al., Cultures of human embryonic stem cells: serum replacement medium or serum-containing media and the effect of basic fibroblast growth factor. Reprod Biomed Online, 2004. 9(3): p. 330-7.

9. Richards, M., et al., Human feeders support prolonged undifferentiated growth of human inner cell masses and embryonic stem cells. Nat Biotechnol, 2002. 20(9): p. 933-6.

10. Rajala, K., et al., Testing of nine different xeno-free culture media for human embryonic stem cell cultures. Hum Reprod, 2007. 22(5): p. 1231-8.

11. Johansson, B. M. and M. V. Wiles, Evidence for involvement of activin A and bone morphogenetic protein 4 in mammalian mesoderm and hematopoietic development. Mol Cell Biol, 1995. 15(1): p. 141-51.

12. Wiles, M. V. and B. M. Johansson, Embryonic stem cell development in a chemically defined medium. Exp Cell Res, 1999. 247(1): p. 241-8.

13. Price P, G. M., Tilkins M L, Embryonic stem cell serum replacer, in WIPO. 1998, Gibco Life Technologies: PCT.

14. Amit, M., et al., Clonally derived human embryonic stem cell lines maintain pluripotency and proliferative potential for prolonged periods of culture. Dev Biol, 2000. 227(2): p. 271-8.

15. Abeyta, M. J., et al., Unique gene expression signatures of independently-derived human embryonic stem cell lines. Hum Mol Genet, 2004. 13(6): p. 601-8.

16. Inzunza, J., et al., Derivation of human embryonic stem cell lines in serum replacement medium using postnatal human fibroblasts as feeder cells. Stem Cells, 2005. 23(4): p. 544-9.

17. Genbacev, O., et al., Serum-free derivation of human embryonic stem cell lines on human placental fibroblast feeders. Fertil Steril, 2005. 83(5): p. 1517-29.

18. Skottman, H., et al., Unique gene expression signature by human embryonic stem cells cultured under serum-free conditions correlates with their enhanced and prolonged growth in an undifferentiated stage. Stem Cells, 2006. 24(1): p. 151-67.

19. Amit, M., et al., Feeder layer- and serum-free culture of human embryonic stem cells. Biol Reprod, 2004. 70(3): p. 837-45.

20. Williams, R. L., et al., Myeloid leukaemia inhibitory factor maintains the developmental potential of embryonic stem cells. Nature, 1988. 336(6200): p. 684-7.

21. Smith, A. G., et al., Inhibition of pluripotential embryonic stem cell differentiation by purified polypeptides. Nature, 1988. 336(6200): p. 688-90.

22. Ying, Q. L., et al., BMP induction of Id proteins suppresses differentiation and sustains embryonic stem cell self-renewal in collaboration with STAT3. Cell, 2003. 115(3): p. 281-92.

23. Xu, R. H., et al., BMP4 initiates human embryonic stem cell differentiation to trophoblast. Nat Biotechnol, 2002. 20(12): p. 1261-4.

24. Xu, R. H., et al., Basic FGF and suppression of BMP signaling sustain undifferentiated proliferation of human ES cells. Nat Methods, 2005. 2(3): p. 185-90.

25. Humphrey, R. K., et al., Maintenance of pluripotency in human embryonic stem cells is STAT3 independent. Stem Cells, 2004. 22(4): p. 522-30.

26. Daheron, L., et al., LIF/STAT3 signaling fails to maintain self-renewal of human embryonic stem cells. Stem Cells, 2004. 22(5): p. 770-8.

27. Xu, C., et al., Feeder-free growth of undifferentiated human embryonic stem cells. Nat Biotechnol, 2001. 19(10): p. 971-4.

28. Rosler, E. S., et al., Long-term culture of human embryonic stem cells in feeder-free conditions. Dev Dyn, 2004. 229(2): p. 259-74.

29. Greber, B., H. Lehrach, and J. Adjaye, Fibroblast growth factor 2 modulates transforming growth factor beta signaling in mouse embryonic fibroblasts and human ESCs (hESCs) to support hESC self-renewal. Stem Cells, 2007. 25(2): p. 455-64.

30. Beattie, G. M., et al., Activin A maintains pluripotency of human embryonic stem cells in the absence of feeder layers. Stem Cells, 2005. 23(4): p. 489-95.

31. Pebay, A., et al., Essential roles of sphingosine-1-phosphate and platelet-derived growth factor in the maintenance of human embryonic stem cells. Stem Cells, 2005. 23(10): p. 1541-8.

32. James, D., et al., TGFbeta/activin/nodal signaling is necessary for the maintenance of pluripotency in human embryonic stem cells. Development, 2005. 132(6): p. 1273-82.

33. Vallier, L., M. Alexander, and R. A. Pedersen, Activin/Nodal and FGF pathways cooperate to maintain pluripotency of human embryonic stem cells. J Cell Sci, 2005. 118(Pt 19): p. 4495-509.

34. Pyle, A. D., L. F. Lock, and P. J. Donovan, Neurotrophins mediate human embryonic stem cell survival. Nat Biotechnol, 2006. 24(3): p. 344-50.

35. Ding, S. and P. G. Schultz, A role for chemistry in stem cell biology. Nat Biotechnol, 2004. 22(7): p. 833-40.

36. Chen, S., S. Hilcove, and S. Ding, Exploring stem cell biology with small molecules. Mol Biosyst, 2006. 2(1): p. 18-24.

37. Liu, J., et al., A small-molecule agonist of the Wnt signaling pathway. Angew Chem Int Ed Engl, 2005. 44(13): p. 1987-90.

38. Qi, X., et al., BMP4 supports self-renewal of embryonic stem cells by inhibiting mitogen-activated protein kinase pathways. Proc Natl Acad Sci USA, 2004. 101(16): p. 6027-32.

39. Sato, N., et al., Maintenance of pluripotency in human and mouse embryonic stem cells through activation of Wnt signaling by a pharmacological GSK-3-specific inhibitor. Nat Med, 2004. 10(1): p. 55-63.

40. Chen, S., et al., Self-renewal of embryonic stem cells by a small molecule. Proc Natl Acad Sci USA, 2006. 103(46): p. 17266-71.

41. Miyabayashi, T., et al., Wnt/beta-catenin/CBP signaling maintains long-term murine embryonic stem cell pluripotency. Proc Natl Acad Sci USA, 2007. 104(13): p. 5668-73.

42. Martin, M. J., et al., Human embryonic stem cells express an immunogenic nonhuman sialic acid. Nat Med, 2005. 11(2): p. 228-32.

43. Ludwig, T. E., et al., Derivation of human embryonic stem cells in defined conditions. Nat Biotechnol, 2006. 24(2): p. 185-7.

44. Richards, M., et al., Comparative evaluation of various human feeders for prolonged undifferentiated growth of human embryonic stem cells. Stem Cells, 2003. 21(5): p. 546-56.

45. Cheng, L., et al., Human adult marrow cells support prolonged expansion of human embryonic stem cells in culture. Stem Cells, 2003. 21(2): p. 131-42.

46. Amit, M., et al., Human feeder layers for human embryonic stem cells. Biol Reprod, 2003. 68(6): p. 2150-6.

47. Hovatta, O., et al., A culture system using human foreskin fibroblasts as feeder cells allows production of human embryonic stem cells. Hum Reprod, 2003. 18(7): p. 1404-9.

48. Choo, A. B., et al., Expansion of pluripotent human embryonic stem cells on human feeders. Biotechnol Bioeng, 2004. 88(3): p. 321-31.

49. Stojkovic, P., et al., An autogeneic feeder cell system that efficiently supports growth of undifferentiated human embryonic stem cells. Stem Cells, 2005. 23(3): p. 306-14.

50. Klimanskaya, I., et al., Human embryonic stem cells derived without feeder cells. Lancet, 2005. 365(9471): p. 1636-41.

51. Stojkovic, P., et al., Human-serum matrix supports undifferentiated growth of human embryonic stem cells. Stem Cells, 2005. 23(7): p. 895-902.

52. Mallon, B. S., et al., Toward xeno-free culture of human embryonic stem cells. Int J Biochem Cell Biol, 2006. 38(7): p. 1063-75.

53. Xu, C., et al., Basic fibroblast growth factor supports undifferentiated human embryonic stem cell growth without conditioned medium. Stem Cells, 2005. 23(3): p. 315-23.

54. Yu, J., et al., Induced pluripotent stem cell lines derived from human somatic cells. Science, 2007. 318(5858): p. 1917-20.

55. Ludwig, T. E., et al., Feeder-independent culture of human embryonic stem cells. Nat Methods, 2006. 3(8): p. 637-46.

56. Yao, S., et al., Long-term self-renewal and directed differentiation of human embryonic stem cells in chemically defined conditions. Proc Natl Acad Sci USA, 2006. 103(18): p. 6907-12.

57. Li, Y. J., et al., Hydrogels as artificial matrices for human embryonic stem cell self-renewal. J Biomed Mater Res A, 2006. 79(1): p. 1-5.

58. Anderson, D. G., S. Levenberg, and R. Langer, Nanoliter-scale synthesis of arrayed biomaterials and application to human embryonic stem cells. Nat Biotechnol, 2004. 22(7): p. 863-6.

59. Wichterle, H., et al., Directed differentiation of embryonic stem cells into motor neurons. Cell, 2002. 110(3): p. 385-97.

60. Isacson, O., Problems and solutions for circuits and synapses in Parkinson's disease. Neuron, 2004. 43(2): p. 165-8.

61. Kim, J. H., et al., Dopamine neurons derived from embryonic stem cells function in an animal model of Parkinson's disease. Nature, 2002. 418(6893): p. 50-6.

62. Shapiro, A. M. and J. R. Lakey, Future trends in islet cell transplantation. Diabetes Technol Ther, 2000. 2(3): p. 449-52.

63. Facchinetti, F., et al., Neurotrophic factors in the therapy of Parkinson's disease. Funct Neurol, 2001. 16(1): p. 45-56.

64. Wente, M. N., et al., Blockade of the chemokine receptor CXCR2 inhibits pancreatic cancer cell-induced angiogenesis. Cancer Lett, 2006. 241(2): p. 221-7.

65. Kyriakides, T. R., et al., The CC chemokine ligand, CCL2/MCP1, participates in macrophage fusion and foreign body giant cell formation. Am J Pathol, 2004. 165(6): p. 2157-66.

66. Quelle, F. W., et al., JAK2 associates with the beta c chain of the receptor for granulocyte-macrophage colony-stimulating factor, and its activation requires the membrane-proximal region. Mol Cell Biol, 1994. 14(7): p. 4335-41.

67. Taga, T. and T. Kishimoto, Signaling mechanisms through cytokine receptors that share signal transducing receptor components. Curr Opin Immunol, 1995. 7(1): p. 17-23.

68. Corcoran, A. E., et al., The interleukin-7 receptor alpha chain transmits distinct signals for proliferation and differentiation during B lymphopoiesis. Embo J, 1996. 15(8): p. 1924-32.

69. Sato, N., et al., Signal transduction by the high-affinity GM-CSF receptor: two distinct cytoplasmic regions of the common beta subunit responsible for different signaling. Embo J, 1993. 12(11): p. 4181-9.

70. Pelus, L. M. and S. Fukuda, Peripheral blood stem cell mobilization: the CXCR2 ligand GRObeta rapidly mobilizes hematopoietic stem cells with enhanced engraftment properties. Exp Hematol, 2006. 34(8): p. 1010-20.

71. Carr, M. W., et al., Monocyte chemoattractant protein 1 acts as a T-lymphocyte chemoattractant. Proc Natl Acad Sci USA, 1994. 91(9): p. 3652-6.

I claim:

1. A growth factor supplement for stem cell culture media comprising a combination of interleukin-5, Interleukin-6 and interleukin-7.

2. A growth factor supplement according to claim 1, further comprising granulocyte macrophage colony stimulating factor.

3. A growth factor supplement according to claim 2, further comprising GRO gamma/CXCL3.

4. A growth factor supplement according to claim 3, further comprising CCL2/MCP-1.

5. The supplement of claims 1, 2, 3, or 4, wherein the stem cells are IPS cells.

6. The supplement of claims 1, 2, 3, or 4, wherein the stem cells are adult stem cells.

7. The supplement of claims 1, 2, 3, or 4, wherein the stem cells are embryonic stem cells.

8. The supplement of claims 1, 2, 3, or 4, wherein the stem cells are neonatal stem cells.

9. A stem cell culture media comprising a growth factor supplement that is a combination of interleukin-5, Interleukin-6 and interleukin-7.

10. A stem cell culture media according to claim 9, further comprising granulocyte macrophage colony stimulating factor.

11. A stem cell culture media according to claim 10, further comprising GRO gamma/CXCL3.

12. A stem cell culture media according to claim 11, further comprising CCL2/MCP-1.

13. A stem cell culture media according to claims 9, 10, 11 or 12, wherein the stem cells are IPS cells.

14. A stem cell culture media according to claims 9, 10, 11 or 12, wherein the stem cells are adult stem cells.

15. A stem cell culture media according to claims 9, 10, 11 or 12, wherein the stem cells are embryonic stem cells.

16. A stem cell culture media according to claims 9, 10, 11 or 12, wherein the stem cells are neonatal stem cells.

17. A method for growing stem cells comprising the step of culturing a stem cell in a growth media supplemented with a growth factor supplement of claims 1, 2, 3 or 4.

18. The method of claim 17, wherein the supplemented growth media enhances stem cell plating efficiency.

19. The method of claim 17, wherein the supplemented growth media enhances stem cell plating efficiency proliferation.

20. The method of claim 17, wherein the supplemented growth media prolongs media effectiveness for stem cell growth by at least two-fold.

21. The method of claim 17, wherein the stem cells are IPS cells.

22. The method of claim 17, wherein the stem cells are adult stem cells.

23. The method of claim 17, wherein the stem cells are embryonic stem cells.

24. The method of claim 17, wherein the stem cells are neonatal stem cells.

25. A method for growing stem cells comprising the step of culturing a stem cell in a supplemented growth media of claims 9, 10, 11 or 12.

26. The method of claim 25, wherein the supplemented growth media enhances stem cell plating efficiency.

27. The method of claim 25, wherein the supplemented growth media enhances stem cell plating efficiency proliferation.

28. The method of claim 25, wherein the supplemented growth media prolongs media effectiveness for stem cell growth by at least two-fold.

29. The method of claim 25, wherein the stem cells are IPS cells.

30. The method of claim 25, wherein the stem cells are adult stem cells.

31. The method of claim 25, wherein the stem cells are embryonic stem cells.

32. The method of claim 25, wherein the stem cells are neonatal stem cells.
